(19) United States
(12) Patent Application Publication
(10) Pub. No.: US 2025/0195103 A1
(43) Pub. Date: Jun. 19, 2025

(54) METHODS, SYSTEMS AND APPARATUSES FOR TRANSSEPTAL PROCEDURES

(71) Applicant: CASE WESTERN RESERVE UNIVERSITY, Cleveland, OH (US)

(72) Inventors: Andrew Rollins, Cleveland, OH (US); Xiaowei Zhao, Cleveland, OH (US)

(21) Appl. No.: 18/849,099

(22) PCT Filed: Mar. 22, 2023
(86) PCT No.: PCT/US2023/015952

§ 371 Date: Sep. 20, 2024

Related U.S. Application Data
(60) Provisional application No. 63/322,471, filed on Mar. 22, 2022.

(51) Int. Cl. A61B 17/34; A61B 17/00; A61B 90/00; G06T 7/60

(52) U.S. Cl. CPC A61B 17/34; A61B 17/00234; A61B 90/36; G06T 7/60; A61B 2017/00247; A61B 2017/00305; A61B 2090/3735; G06T 2207/10101; G06T 2207/30048

(57) ABSTRACT

A catheter device (10) includes an elongate tubular sheath (16) having spaced apart proximal and distal ends and a lumen (20, 22) extending through the elongate tubular sheath. An elongate needle (14) is within the lumen (20. 22) and terminating in a distal end portion adjacent the distal end of the elongate tubular sheath. The distal end portion of the needle (14) has a central axis extending through the tip portion. An optical probe (12) extends within the lumen and terminating in a distal end adjacent the distal end of the elongate tubular sheath. The distal end of the optical probe (12) is configured to provide an image field of view (62) having a known position relative to the distal end portion of the needle (14).

### GOVERNMENT FUNDING

This invention was made with government support under HL149369 awarded by The National Institutes of Health. The government has certain rights in the invention.

### TECHNICAL FIELD

This disclosure relates to methods, systems and apparatuses for transseptal procedures.

### BACKGROUND

The transseptal puncture (TSP) procedure was invented in the late 1950s to measure the intracardiac pressure of the left atrium (LA). Nowadays, it is commonly conducted in both pediatric and adult patients for procedures which need percutaneous access to the LA, such as for catheter ablation (CA) for atrial fibrillation (AF), paravalvular leakage repair, patent foramen ovale (PFO) closure, percutaneous mitral valve replacement, atrial appendage closure, and left ventricular assist device positioning. In a TSP procedure, a needle is introduced into the right atrium and used to puncture the interatrial septum (IAS) at its thinnest location, the fossa ovalis (FO), typically under the guidance of fluoroscopy and/or intracardiac echocardiography (ICE). Although the procedure is relatively safe, the precision of the puncture site is important to reduce the risk of complications, such as perforation of the heart or large vessels, as well as the delivery of devices into the desired portion of the LA and therefore facilitate the whole procedure. With current TSP guidance, fluoroscopy exposes patients to ionizing radiation and ICE requires a large vascular sheath, which may be challenging in patients with smaller hearts (e.g., pediatric patients). A simplified way to confirm that the catheter tip is correctly positioned at the FO is needed.

### SUMMARY

This disclosure relates to methods, systems and apparatuses for transseptal procedures.

One example relates to a multi-function catheter device. The catheter device includes an elongate tubular sheath having spaced apart proximal and distal ends and a lumen extending through the elongate tubular sheath. An elongate needle is within the lumen and terminates in a distal end portion adjacent the distal end of the elongate tubular sheath, in which the distal end portion of the needle has a central axis extending through the tip portion.

An optical probe extends within the lumen and terminates in a distal end adjacent the distal end of the elongate tubular sheath, in which the distal end of the optical probe is configured to provide optical radiation and receive reflected and/or backscattered optical signals for an image field of view having a known spatial arrangement with respect to the distal end portion of the needle.

Another example relates to a system that includes a catheter and an imaging system. The catheter includes an elongate tubular sheath having spaced apart proximal and distal ends and a lumen extending through the elongate tubular sheath. An elongate needle is within the lumen and terminates in a distal end portion adjacent the distal end of the elongate tubular sheath, and the distal end portion of the needle has a central axis extending through the tip portion. An optical probe extends within the lumen and terminates in a distal end adjacent the distal end of the elongate tubular sheath, in which the distal end of the optical probe is configured to provide optical radiation and receive reflected and/or backscattered optical signals for an image field of view having a known spatial arrangement with respect to the distal end portion of the needle. The imaging system includes a light source coupled to the proximal end of the optical probe. The imaging system also includes an optical detector coupled to the optical probe and configured to detect an optical signal from the optical probe and provide a detector signal representative of light reflected and/or scattered from at least one object within the image field of view.

Another example relates to a method of using a catheter. The method includes positioning a distal end of a catheter in a right atrium of a heart, in which the catheter includes an elongate tubular sheath having spaced apart proximal and distal ends and a lumen extending through the elongate tubular sheath. The catheter also includes an elongate needle within the lumen and terminating in a distal end portion adjacent the distal end of the elongate tubular sheath, in which the distal end portion of the needle has a central axis extending through the tip portion. An optical probe extends within the lumen and terminates in a distal end adjacent the distal end of the elongate tubular sheath, in which the distal end of the optical probe is configured to provide optical radiation and receive reflected and/or backscattered optical signals for an image field of view having a known spatial arrangement with respect to the distal end portion of the needle. The method also includes locating a puncture site on the septum based on a detector signal provided by an optical detector coupled to the optical probe, in which the detector signal is representative of light reflected and/or scattered from at least one object within the image field of view responsive to light provided from light source. The method also includes advancing the needle from the catheter to puncture through the septum at the puncture site to provide an access port from the right atrium to a left atrium of the heart.

### BRIEF DESCRIPTION OF THE DRAWINGS

FIG. 1 depicts an example of a multi-function catheter.

FIG. 2 depicts an example of part of an OCT probe.

FIG. 3 depicts an example of an OCT system.

FIG. 4 is a flow diagram showing an example of a method for performing a TSP procedure.

FIGS. 5 and 6 depict an example of a TSP procedure.

FIG. 7A depicts an example OCT image and FIG. 7B is a photograph showing the IAS and FO.

FIGS. 8A and 8B depict other examples of OCT images and FIG. 8C is a photograph showing the IAS and FO.

FIGS. 9A, 9B, 9C and 9D depicts further examples OCT images, in which FIG. 9A is an OCT image generated with a benchtop scanner, FIG. 9B is a photograph showing the IAS and FO generated with a benchtop scanner, and FIGS. 9C and 9D are OCT images generated with an OCT probe.

FIG. 10 depicts an example OCT image differentiating between blood in the left atrium and IAS.

### DETAILED DESCRIPTION

This disclosure provides a multi-function catheter having an elongate body that includes an optical probe and a needle integrated therein. The optical probe is configured to provide an optical radiation (e.g., light in the visible, ultraviolet and/or infrared parts of the electromagnetic spectrum) and receive reflected and backscattered optical signals. The received signals are detected and processed (e.g., using interferometry) to provide one or more images for objects within the image field of view. In an example, the optical probe is an optical coherence tomography (OCT) probe configured to provide the optical radiation to illuminate an optical field of view. The optical radiation can be provided in the form of a beam or other pattern. For example, the optical probe is configured to provide image signals of the IAS during a TSP procedure. The detector and/or associated processor can generate one or more images representative of optical properties of tissue (e.g., cardiac tissue) responsive to illumination by the optical radiation. In the example of an OCT probe or other probes capable of providing optical illumination that penetrates tissue, the probe can acquire images that includes structures below the surface of tissue, including within and through the IAS. Thus, the OCT probe can generate optical images (e.g., real-time images) to visualize walls of the IAS.

A puncture location, such as the FO, can be determined in the tissue based on the image(s) that is generated. For example, the puncture location is determined based on optical properties of the tissue. The optical properties of the tissue can include birefringence, scattering, attenuation and/or heterogeneity. The optical properties can also include dynamics, such as from changes in the optical signals over time. The processor can also be configured to classify objects within the image field of view based on the optical properties, such as to identify the IAS, the fossa ovalis, blood as well as relevant features or attributes of such objects (e.g., cross-sectional thickness, layer architecture of the tissue, tissue properties, and movement of tissue and/or blood). In one example, the processor is configured to identify a target puncture site at the FO based on determining a thickness of the IAS or other tissue from the optical properties (e.g., a distance between respective surfaces of the IAS).

For example, a real-time image can be generated on a display based on the OCT image data. One or more forms of guidance also can be visualized on a display along with the OCT image (e.g., text and/or graphics superimposed on the image and/or adjacent to the image). Thus, a user can identify the FO or a particular target region thereof and advance the needle through the FO into the LA. Once the needle has penetrated through the FO, additional interventions can be performed (e.g., percutaneous mitral valvuloplasty, mitral valve repair, left atrial appendage (LAA) closure, PFO closure, and ablation, etc.). By using a catheter that integrates an OCT probe and a needle, transseptal puncture (TSP) can be performed more efficiently and with reduced risk of complications.

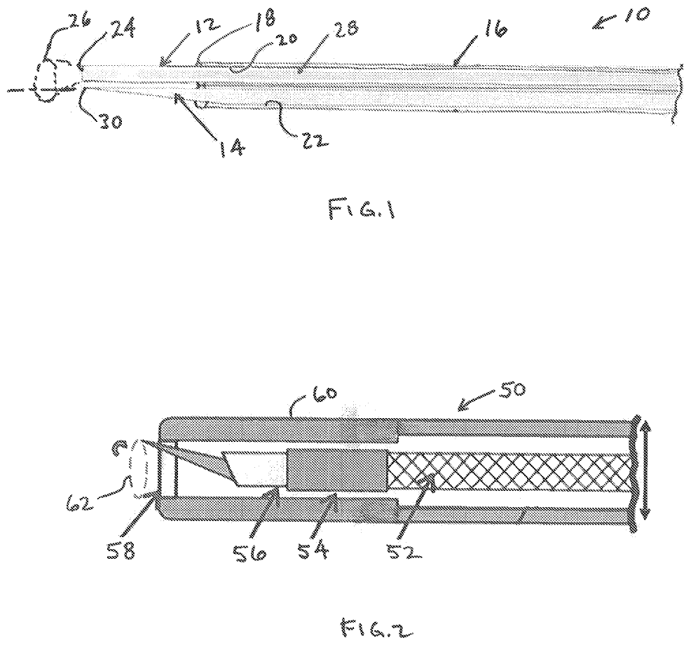

FIG. 1 depicts an example of a multi-function catheter 10 that includes an optical probe 12 and a needle 14 within an elongate tubular sheath 16. The sheath 16 includes a distal end 18 having an opening through which the probe 12 and needle 14 may extend. In an example, the sheath 16 includes separate channels (e.g., parallel lumen) 20 and 22, which extend longitudinally through the sheath 16 to hold each of the probe 12 and the needle 14, respectively. The probe 12 can be configured to move axially as well as rotate within the channel 20. Similarly, the needle 14 can move at least axially within the channel 22. As described herein, the needle 14 can be a transseptal needle having a sharp distal tip adapted for cutting and/or piercing tissue including the IAS. In another example, the needle can be a sharpened guidewire. The needle 14 can be a commercially available transseptal needle, such as is commercially available from Abbott, Johnson & Johnson, Cook Medical and others.

In the example of FIG. 1, the optical probe 12 extends within the channel 20 and terminates in a distal end 24 adjacent the distal end 18 of the elongate tubular sheath 16. Other arrangements for the optical probe 12 and needle can be implemented, such as disclosed herein. For example, the optical probe 12 can be co-axial with the needle 14, such as where the optical probe resides within and is moveable with respect to a central lumen of the needle. In this example, the field of view of the optical probe can include the central axis of the needle, so that when a desired target site is located, the needle can be advanced over the optical probe to contact and puncture the target.

The optical probe 12 is configured to transmit and receive radiation in the optical spectrum. As used herein, the optical spectrum can include light in the visible, ultraviolet and/or infrared parts of the electromagnetic spectrum. As described herein, the optical probe 12 can be configured to provide an image of the tissue structure at a respective location. As used herein, the term image refers to a point or a collection of points of light rays coming from (e.g., reflected) from one or more objects. The optical probe 12 can thus provide an image for one more locations within an image field of view, such as including a 1-dimensional image, 2-dimensional image or 3-dimensional image. The information contained in the image thus varies on the properties of the object and the optical radiation provided by the source that supplied light for transmission from the probe. When the probe 12 is used for identifying a puncture site for the needle 14 within the right atrium, the objects in the field of view 26 that form part or all of the image can include the IAS, aorta, blood or other objects within the field of view.

In many examples that follow, the optical probe 12 is referred to as an optical coherence tomography (OCT) probe. For the example where the optical probe 12 is an OCT probe, the OCT probe configured to perform OCT imaging, such as implementing forward scanning (as shown) or M-mode imaging within an image field of view 26. However, the optical probe 12 is not limited to implementing OCT, and the probe can be configured to implement other forms of optical imaging. For example, the optical probe 12 can be configured to implement laser Doppler imaging, a laser speckle imaging, dynamic light scattering, reflectance spectroscopy or laser polarimetry. Signals derived from any of such optical probes can be analyzed (e.g., by a processor executing instructions) to differentiate FO from IAS and/or aorta. For example, the instructions can be configured to differentiate FO from IAS by detecting blood in close proximity behind the thin FO wall, as blood could shift and broaden the laser Doppler spectrum, alter the temporal fluctuations of the received signal, alter the wavelength spectrum, and/or depolarize the light. As a further example, the instructions can be configured to differentiate between FO, IAS and aorta because the different tissues have different optical properties which could be detected as changes to received signals, such as amplitude, spectrum, and polarization state.

The OCT probe 12 is configured to acquire images of the tissue structure or other objects in front of the sheath distal end 18, which images can be used to guide the transseptal needle as part of the TSP procedure. In an example, the tubular structures that provide respective channels 20 and 22 are side by side and fixed to each other (e.g., by an adhesive, ultrasonic welding, heat joint, friction fitting, or other joining method). The distal end 24 of the OCT probe 12 can be arranged and configured to provide the image field of view 26 that has a known spatial position with respect to the distal end portion of the needle 14. In an example, image field of view 26 substantially aligns with or overlaps with a long central axis of a distal end portion 30 of the needle 14. That is, the OCT probe 12 and the needle 14 can approximately aim at the same location at the same time. Thus, by advancing the needle 14 axially through the channel 22, the distal end portion (e.g., tip) 30 of the needle can intersect a given location within the field of view 26 of the OCT probe 12, which given location can be a desired target puncture location in the FO. In other examples, there can be a known radial spatial offset between the long central axis of the needle 14 and the field of view. Also, the field of view can extend an area that is smaller than the target site. For example, the field of view for an M-scan can be the area of a beam, whereas the field of view for a forward circular scanning OCT probe can have a larger area.

In the example of FIG. 1, the OCT probe 12 includes an optical fiber 28 that extends longitudinally through the channel 20. To reduce the diameter of the OCT probe 12. the probe can be implemented as an all-fiber OCT probe. For example, the probe can include a combination of any or all of a single mode fiber, coreless fiber, multimode fiber, graded-index (GRIN) fiber, and/or three-dimensional (3D) printed lens on an optical fiber. In other examples (see, e.g., FIG. 2) the OCT probe 12 can be implemented in other configurations. In the all-fiber example, the optical fiber 28 is configured to transmit light from the proximal side (not shown) to the distal end 24 of the fiber 28 and deliver it onto target tissue within the field of view 26. The same optical fiber 28 is configured to receive back scattered light from one or more targets within a field of view of the probe 12, which can be implemented concurrently with transmitting light onto a target. In another example, the OCT probe 12 can be a common-path OCT probe configured to implement common-path OCT imaging, such as disclosed in Fu X. Patel D, Zhu H, MacLennan G, Wang Y T, Jenkins M W, Rollins A M, “Miniature forward-viewing common-path OCT probe for imaging the renal pelvis,” Biomed Opt Express. 2015 Mar. 6; 6 (4):1164-71, which is incorporated herein by reference in its entirety.

As a further example, the catheter 10 is configured so the sheath 16 is steerable. For example, the catheter 10 includes one or more pull-wires (not shown) coupled to the sheath configured to deflect the distal end portion of the sheath 16 in one or more directions to facilitate positioning the distal end of the sheath into the right atrium and at the target puncture location—the FO. For example, the sheath 16, which contains the OCT probe 12 and the needle 14 therein, can be advanced through the right femoral artery and steered along a respective trajectory into the right atrium. During such positioning, the OCT probe 12 and needle 14 can be retracted to reside within the sheath channels 20 and 22. An OCT image can be generated and displayed on display device (e.g., display screen, augmented or virtual reality headset) to provide guidance based on the OCT imaging data. When OCT guidance indicates the needle is at the desired target location, the needle can be advanced axially from the sheath 16 to make the puncture through the tissue at the target location.

The guidance can include or be derived from a thickness of tissue determined from the OCT image data. The guidance can also include differentiating the FO from the surrounding IAS, as determined based on the image data. In some examples, guidance can also include differentiating the FO from the aorta, such as based on the image data, to reduce the likelihood of inadvertently puncturing the aorta during the procedure. The guidance further can identify a boundary between blood and tissue (e.g., on respective right and/or left sides of the IAS and FO) based on the image data.

In some examples, fluoroscopy can be used to confirm the location of the needle at the distal end of the catheter. In an additional, or alternative example, a three-dimensional mapping system can be configured to localize the catheter and map the catheter in 3D space. The location of the catheter in 3D space can be combined with the OCT image data and/or with information derived from the OCT image data.

FIG. 2 depicts an example of an OCT probe 50. The OCT probe 50 is one example OCT probe that can be used to implement the probe 12 in the catheter 10 of FIG. 1. Other configurations of OCT probes could be used in other examples, including the fiber-only probe mentioned above. In the example of FIG. 2, the probe 50 has an inner body portion that includes a torque coil 52, a ferrule 54 and a lens 56. An optical fiber can reside within the torque coil, and the ferrule can couple the optical fiber to the lens for propagating transmitting and receiving light. The inner body portion or probe 50 (e.g., torque coil 52, ferrule 54, lens 56 and fiber) can be an integrated assembly within a housing 60 (e.g., an outer sheath), and the probe can is configured to rotate about a longitudinal axis relative to the housing. The probe 50 also includes a window 58 mounted at a distal end of the housing 60, which can be spaced axially from an inner surface of the lens 56. The window 58 can be formed of glass or other material transparent to the wavelength(s) of light transmitted from a distal end of the lens 56. The housing 60 can be implemented as a monolithic outer sheath structure that extends between proximal and distal ends thereof or, in other examples, it can be formed by connecting two or more parts axially together along its length.

For example, the OCT probe 50 is a forward scanning probe that is configured to rotate the body portion about its long axis. In an example, the probe body 50, including its constituent optical parts and its housing 60, can also move axially within the catheter channel 20 or 20 (e.g., toward or away from the distal end 18 to transmit light at a desired wavelength by covering an image field of view 62 in one or more dimensions. For instance, the probe 50 can move together with its housing 60 axially back and forth to contact adjacent objects (e.g., tissue within the heart). The OCT probe 50 is also configured to receive backscattered light at the lens and provide the received light to an interferometer (e.g., a Michelson interferometer) configured to generate OCT scan data based on the received backscattered light. A processor (e.g., a digital signal processor or a computing device (not shown)) is configured to receive and process the OCT image scan data (representing the OCT image scans) provided by the interferometer and generate OCT images for tissue and other objects within the image field of view 62. The processor can also be programmed to determine optical properties of and/or classify tissue based on the OCT images, such as described herein.

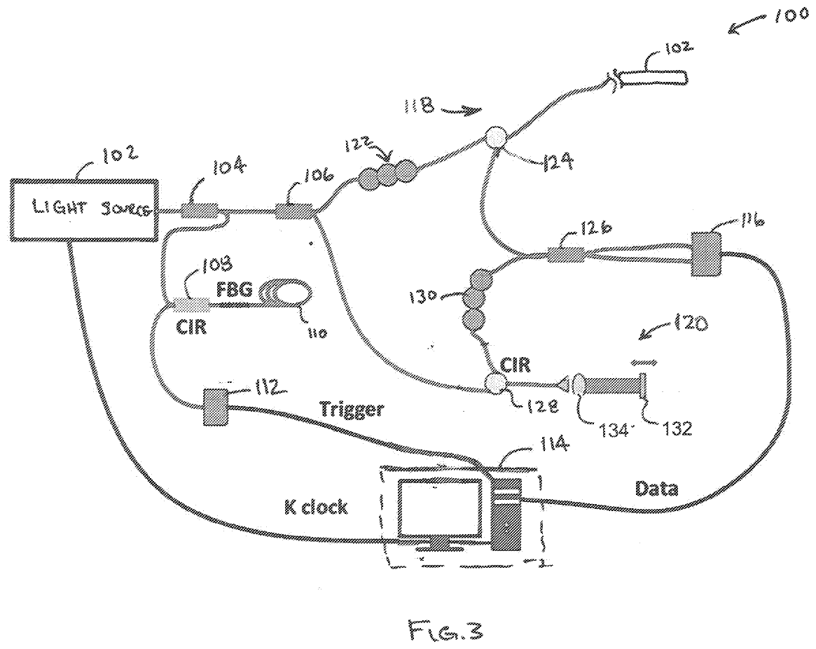

FIG. 3 depicts an example of OCT system 100 configured to implement OCT imaging. Other types and configurations of OCT systems can be used to implement OCT imaging. The system 100 includes an OCT probe, schematically shown at 102. The probe 102 can be implemented, for example, by the probe 12 or 50. A light source (e.g., a swept laser) 104 is configured to provide light at a desired wavelength to a fiber coupler 106 (e.g., a 99/1 coupler), which provides the light to another fiber coupler 106 (e.g., a 90/10 coupler) and a circulator 108. The circulator 108 is coupled to a fiber Bragg grating 110. The circulator 108 is also coupled to a detector 112, which is a photodetector configured to detect light from the fiber Bragg grating and provide a trigger signal to a computing apparatus 114 based on the detected light. The computing apparatus 114 is configured to receive a clock signal (e.g., a K-clock signal) generated by the light source. The clock and/or trigger signals can be used by the computing apparatus to control the acquisition of data representing light signals from another detector 116.

The fiber coupler 106 is also coupled to provide light to a sample arm 118 and a reference arm 120. For example, the 90% light output of the coupler 106 is coupled to the sample arm 118, and 10% output of the coupler 106 is coupled to the reference arm 120. In the sample arm 118, light is coupled to the probe 102 through a circulator 124. The circulator 124 also has another output coupled to a fiber coupler 126 to provide received backscattered light from the probe 102.

The fiber coupler (e.g., a fiber beam splitter) 126 also is coupled to receive a reference signal from the reference arm 120. The reference arm 120 includes a circulator 128 which sends light to an optical delay line and sends the reflected light to the fiber coupler 126. For example, the optical delay line includes a mirror 132 that is positioned at a specific optical path length from a lens 134 corresponding to length of the OCT probe, and an optical input coupler 136 receives the light reflected from the mirror 132 and provides the reference light to a port of the circulator 128, which supplies the reference light to an input of the fiber coupler 126. The combination of reflected light from the sample arm 118 and reference light from the reference arm 120 gives rise to an interference pattern. The detector 116 thus has inputs coupled to receive the interference pattern. The detected signal is converted from analog to digital (AD) form and provided to the computing apparatus 114 for signal and image processing, as described herein.

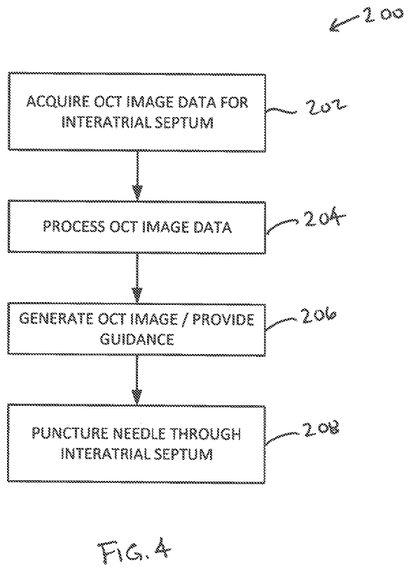

FIG. 4 is a flow diagram showing an example of a method for performing a TSP procedure. The method 200 starts at 202 to acquire OCT images for an IAS. Thus, prior to the method 200, a catheter (e.g., catheter 10), which includes an integrated OCT probe and a needle, is advanced to the right atrium through a minimally invasive procedure, such as through a femoral vein, to the inferior vena cava and into the right atrium, as is known in the art. The placement into the right atrium can be guided by fluoroscopy or another imaging modality. Once the distal end of the catheter is positioned with the right atrium, the distal end can be manipulated (e.g., by steering with one or more pull wires) to place the IAS within the field of view of the OCT probe, such as shown in FIG. 5. OCT images can be generated and processed at 204. As described herein, the processor can be configured to implement image processing techniques applied to the image data as well as analyzing the images, determining optical properties of tissue in the images and/or classifying tissue based on the image data and the optical properties. For example, optical properties can be derived from the OCT images, including birefringence, scattering, attenuation and/or heterogeneity. Tissue and other objects can also be classified based on the optical properties, such as to identify blood, aorta and the IAS, including specifically the FO. The optical properties can be determined based on a time variance detected in the OCT signals, such as by using Doppler OCT or optical coherence tomography angiography (OCT-A).

In some examples, the processing at 204 can include executing instructions that implement a trained deep machine learning model that can be applied to the optical image data (e.g., produced by the optical probe and/or following image processing) to determine one or more tissue properties of the tissue objects in the image data. For example, the machine learning model can be implemented as a convolutional neural network (CNN) having a plurality of layers trained to analyze some or all of the image data frames to perform corresponding pattern recognition and regression analysis to classify one or more tissue properties for object(s) in the acquired image(s) and/or differentiate between different parts of the image(s). Other methods can be implemented at 204 to classify various objects (e.g., the septum, the fossa ovalis on the septum, the aorta, blood, or other tissue), to differentiate between blood and tissue, and/or identify boundaries between blood and tissue based on the optical properties.

At 206, the method includes generating an OCT image and/or providing guidance, such as based on the optical properties of classification (at 204). The guidance can include identifying a target puncture site in the displayed image, such as a target site in the FO. For example, the optical properties or time variance determined from the OCT signals can be used to confirm that the distal end of the probe is located at the FO based on the image. Such confirmation can be determined by the user viewing a real-time image and/or based on imaging processing programmed to ascertain that the needle is aligned at a suitable target site.

Alternatively, or additionally, the guidance can be inferred by the user from displayed image, such as by locating the area showing a thinnest region of the IAS, which can include showing movement of blood on the left-atrial side. The displayed image can also be annotated to provide such guidance, such as by labeling a thickness of the IAS (e.g., calculated as a distance between front and back surfaces of the IAS, which have been segmented from the OCT image). In an example, once positioned at the target site, the puncture site can be confirmed through fluoroscopy or another imaging modality. Additionally, or alternatively, the location of the needle within the heart can be guided by a surgical navigation (e.g., 3D mapping) system, such as are commercially available from Medtronic, Johnson & Johnson, Abbott to name a few. For example, the OCT image can be co-registered with the position of the distal end of the probe in the 3D mapping system and used to confirm a desired target puncture site.

Once a desired target puncture site is identified, the method proceeds to 208, in which the need is advanced to puncture through the IAS at the target site, such as shown in FIG. 6. After puncturing through the IAS, a further procedure or procedures can be implemented. For example, a sheath (e.g., the catheter that carried the needle) can be advanced over the needle or a guidewire following the needle into the LA. The sheath can be used to implant an object (e.g., a stent, a valve) into LA, to perform a repair procedure (e.g., LAA closure etc.) or to perform an ablation (e.g., of cardiac tissue in the LA). In an example, the procedure can be performed through the sheath within the catheter that carried the needle, such as through the channel 22 (after the needle has been removed). In another example, another sheath may be introduced, such as along a guidewire placed into the LA through the punctured site.

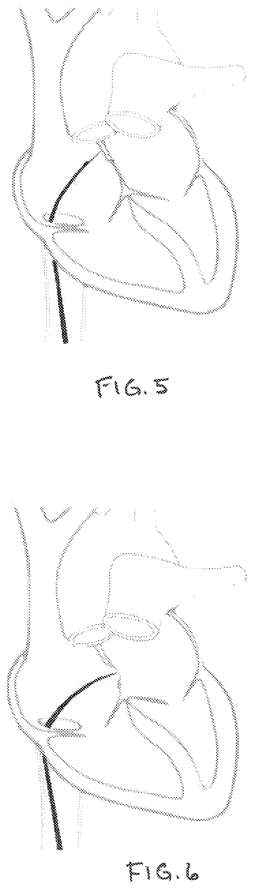

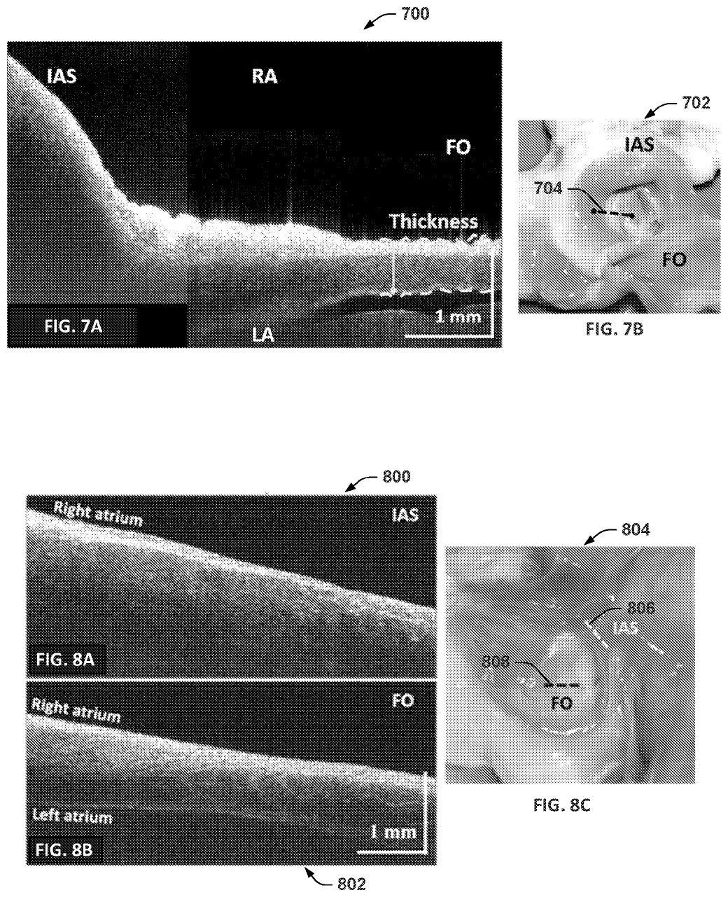

FIG. 7A depicts an example OCT image 700 showing the IAS and FO. The OCT image 700 has also been annotated to show thickness of the FO. FIG. 7B depicts a corresponding optical image (e.g., a photograph) 702 taken externally also showing the IAS and FO, in which the location where the OCT image is taken is along the dashed line, shown at 704.

FIGS. 8A and 8B depict other example OCT images 800 and 802 for different parts of the IAS. FIG. 8C depicts an optical image (e.g., a photograph) 804 taken externally for part of the heart showing the same IAS and FO. Specifically, FIG. 8A shows the OCT image 800 for the IAS spaced from the FO, taken along the dashed line in FIG. 8C, shown at 806. Similarly, FIG. 8B shows the OCT image 802 for the FO, taken along the dashed line in FIG. 8C, shown at 808. Thus, by comparing different images, a surgeon or other individual performing the procedure can easily determine that the IAS tissue in FIG. 8A is thicker (e.g., about two times or more) than the FO tissue in FIG. 8B. Such comparison enables the user to readily ascertain the relative thickness of tissue (in real-time images that are displayed) and infer a suitable thin target site for puncture, namely at the FO. As mentioned, because the spatial location of the needle tip is known with sufficient accuracy relative to the field of view presented in the image (e.g., it can be within the FOV or spaced from the FOV by a known distance), the user can advance the needle to puncture the FO with confidence.

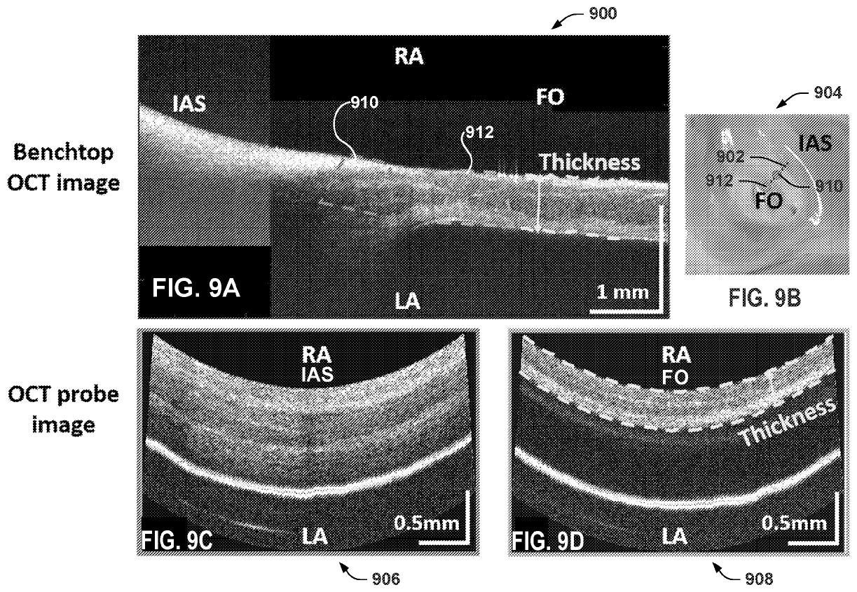

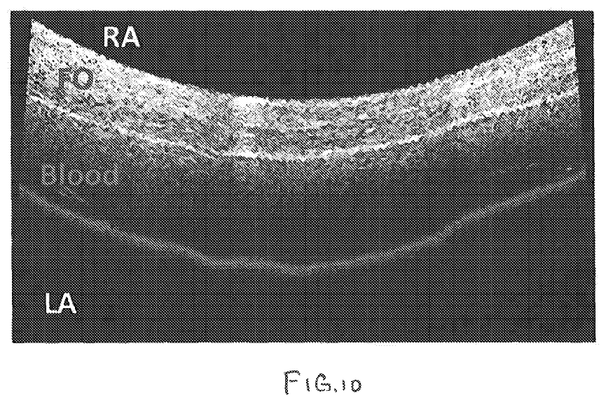

FIGS. 9A, 9B, 9C and 9D depicts further examples OCT images showing the IAS and FO. For example, FIG. 9A shows an OCT image 900 acquired using a benchtop system (shown in FIG. 9A). The OCT image shows the IAS, FO and their relative position to the RA and LA, as taken by the benchtop system along dashed line 902 in optical image (e.g., photograph) 904 of heart tissue at the IAS. FIGS. 9C and 9D depict respective OCT images 906 and 908 for different areas of tissue acquired using an OCT probe, such as described herein. The region of tissue shown in the image 906 of FIG. 9C is represented at 910 in each of FIGS. 9A and 9B. The region of tissue shown in the image 908 of FIG. 9D, which is closer to the center of the FO, is represented at 912 in each of FIGS. 9A and 9B.

FIG. 10 depicts an example in vivo OCT image 1000 taken from an OCT probe, such as disclosed herein. In the example of FIG. 10, the OCT image visually differentiates between IAS tissue and blood based on their respective optical properties, which provides further guidance in identifying a target site (e.g., for TSP).

From the above description of the invention, those skilled in the art will perceive improvements, changes and modifications. Such improvements, changes and modifications within the skill of the art are intended to be covered by this application, including the appended claims. Accordingly, the invention is intended to embrace all such alterations, modifications, and variations that fall within the scope of this application, including the appended claims.

Where the disclosure or claims recite “a,” “an,” “a first,” or “another” element, or the equivalent thereof, it should be interpreted to include one or more than one such element, neither requiring nor excluding two or more such elements.

In this application, the term “couple” or “couples” means either an indirect or direct connection. Thus, if a first device couples to a second device, that connection may be through a direct connection or through an indirect connection via other devices and connections. For example, if device A generates a signal to control device B to perform an action, in a first example device A is coupled to device B, or in a second example device A is coupled to device B through intervening component C if intervening component C does not substantially alter the functional relationship between device A and device B such that device B is controlled by device A via the control signal generated by device A.

Additionally, as used herein, the term “includes” means includes but not limited to, the term “including” means including but not limited to. The term “based on” means based at least in part on. All references, publications, and patents cited in the present application are herein incorporated by reference in their entirety. Unless otherwise stated, in this application, “about,” “approximately” or “substantially” preceding a value means +/−5 percent (5%) of the stated value. For example, “substantially parallel” means being within +/−4.5 degrees of exactly parallel, and “substantially orthogonal” means being within +/−4.5 degrees of exactly orthogonal.

1. A catheter device, comprising:
an elongate tubular sheath having spaced apart proximal and distal ends and a lumen extending through the elongate tubular sheath;
an elongate needle within the lumen and terminating in a distal end portion, including a tip portion, adjacent the distal end of the elongate tubular sheath, the distal end portion of the needle having a central axis extending through the tip portion; and
an optical probe extending within the lumen and terminating in a distal end adjacent the distal end of the elongate tubular sheath, in which the distal end of the optical probe is configured to provide optical radiation and receive reflected and/or backscattered optical signals for an image field of view having a known spatial arrangement with respect to the distal end portion of the needle.

2. The catheter device of claim 1, wherein the lumen includes first and second channels extending coextensively within the lumen, the elongate needle being located within the first channel of the lumen, and the optical probe being located within the second channel of the lumen.

3. The catheter device according to claim 1, wherein the optical probe further comprises at least one optical fiber.

4. The catheter device according to claim 1, wherein the sheath comprises a steerable sheath.

5. The catheter device of claim 1, wherein the optical probe is located within a respective lumen of the needle and the needle is configured to move at least axially relative to the optical probe.

6. The catheter device according to claim 1, wherein the needle is a transseptal needle.

7. The catheter device of according to claim 1, wherein the optical probe is an optical coherence tomography (OCT) probe.

8. The catheter device of claim 1, further comprising:
a light source coupled to the proximal end of the optical probe; and
an optical detector coupled to the optical probe and configured to detect an optical signal from the optical probe and provide a detector signal representative of light reflected and/or scattered from at least one object within the image field of view.

9. A system, comprising:
a catheter, comprising:
an elongate tubular sheath having spaced apart proximal and distal ends and a lumen extending through the elongate tubular sheath;
an elongate needle within the lumen and terminating in a distal end portion, including a tip portion, adjacent the distal end of the elongate tubular sheath, the distal end portion of the needle having a central axis extending through the tip portion; and
an optical probe extending within the lumen and terminating in a distal end adjacent the distal end of the elongate tubular sheath, in which the distal end of the optical probe is configured to provide optical radiation and receive reflected and/or backscattered optical signals for an image field of view having a known spatial arrangement with respect to the distal end portion of the needle;
an imaging system comprising:
a light source coupled to the proximal end of the optical probe; and
an optical detector coupled to the optical probe and configured to detect an optical signal from the optical probe and provide a detector signal representative of light reflected and/or scattered from at least one object within the image field of view.

10. The system of claim 9, wherein the optical probe is an optical coherence tomography (OCT) probe.

11. The system according to claim 9, further comprising a processor configured to analyze the detector signal to determine a thickness of tissue based on calculating a distance between proximal and distal boundaries of the tissue.

12. The system of claim 11, wherein the processor is configured to determine a puncture location in the tissue based on the thickness.

13. The system according to claim 9, further comprising a processor configured to analyze the detector signal to determine optical properties of the at least one object within the image field of view, and wherein the processor is configured to identify a puncture location in the tissue based on optical properties of the tissue.

14. The system of claim 13, wherein the optical properties of the tissue include birefringence, scattering, attenuation and/or heterogeneity.

15. The system according to claim 9-or 10, further comprising a processor configured to analyze the detector signal to determine optical properties of the at least one object within the image field of view, and wherein the processor is configured to determine a thickness of tissue based on the optical properties of the tissue and/or a time variance of the optical signals.

16. The system according to claim 15, wherein the processor is configured to classify the at least one object based on the optical properties.

17. The system of claim 16, wherein the classification of the at least one object includes at least one of identifying a septum, identifying a fossa ovalis on the septum, identifying an aorta, identifying blood, identifying tissue, differentiating between blood and tissue, and identifying boundaries between blood and tissue.

18. The system according to claim 15, wherein the processor is configured to generate an image based on the detector signal and provide the image to a display.

19. The system of claim 18, wherein the processor is further configured to analyze the image and to classify features in the image.

20. The system of claim 19, wherein the processor is further configured to filter and enhance the image that is displayed based on the features classified in the image.

21. The system according to claim 9, further comprising:
a localization system configured to track a location of the catheter in spatial coordinates and to generate spatial map data representative of the location of the catheter over time; and
a processor further configured to generate a visualization of the a spatial map based on the spatial map data and to identify structures in the visualization based on the detector signal and the spatial map data.

22. A method of using a catheter, the method comprising:
positioning a distal end of the catheter in a right atrium of a heart, wherein the catheter comprises:
an elongate tubular sheath having spaced apart proximal and distal ends and a lumen extending through the elongate tubular sheath;
an elongate needle within the lumen and terminating in a distal end portion, including a tip portion, adjacent the distal end of the elongate tubular sheath, the distal end portion of the needle having a central axis extending through the tip portion; and
an optical probe extending within the lumen and terminating in a distal end adjacent the distal end of the elongate tubular sheath, in which the distal end of the optical probe is configured to provide optical radiation and receive reflected and/or backscattered optical signals for an image field of view having a known spatial arrangement with respect to the distal end portion of the needle;
locating a puncture site on a septum of the heart based on a detector signal provided by an optical detector coupled to the optical probe, in which the detector signal is representative of light reflected and/or scattered from at least one object within the image field of view responsive to light provided from light source; and
advancing the needle from the catheter to puncture through the septum at the puncture site to provide an access port from the right atrium to a left atrium of the heart.

23. The method of claim 22, further comprising:
advancing the catheter or other device within the catheter through the access port; and
performing an intervention in the left atrium.

24. The method according to claim 22, wherein prior to advancing the needle, the method includes confirming the puncture site location using a medical imaging modality.
